(19) United States
(12) Patent Application Publication
(10) Pub. No.: US 2025/0134395 A1
(43) Pub. Date: May 1, 2025

(54) BIOMARKER FOR PULMONARY HYPERTENSION DETECTION IN HEART FAILURE

(71) Applicant: Cardiac Pacemakers, Inc., St. Paul, MN (US)

(72) Inventors: Mojgan Goftari, Shoreview, MN (US); Bin Mi, Arden Hills, MN (US); Viktoria A. Averina, Shoreview, MN (US); Pramodsingh Hirasingh Thakur, Woodbury, MN (US)

(21) Appl. No.: 18/904,912

(22) Filed: Oct. 2, 2024

Related U.S. Application Data
(60) Provisional application No. 63/546,073, filed on Oct. 27, 2023.

(51) Int. Cl. A61B 5/0205; A61B 5/00; A61B 5/11; A61B 7/04

(52) U.S. Cl. CPC A61B 5/0205; A61B 5/0031; A61B 5/686; A61B 5/7282; A61B 5/7285; A61B 5/746; A61B 5/1118; A61B 7/04

(57) ABSTRACT

Systems and methods are disclosed to an ambulatory medical device. The ambulatory medical device comprising an activity sensor configured to produce an activity signal representative of activity level of a patient, a heart sound sensor configured to produce a heart sound signal, and a control circuit. The control circuit is configured to detect a change in activity level of the patient using the activity signal, enable monitoring of the heart sound signal in response to detecting the change in activity level, measure intensity of an S3 heart sound in the heart sound signal, and produce an indication of pulmonary hypertension (PH) when the measured intensity of the S3 heart sound exceeds a PH detection threshold intensity.

### CLAIM OF PRIORITY

This application claims the benefit of U.S. Provisional Application No. 63/546,073, filed on Oct. 27, 2023, which is hereby incorporated by reference in its entirety.

### TECHNICAL FIELD

This document relates generally to medical devices and more particularly to systems, methods, and devices for automatic detection of pulmonary hypertension of a patient or subject.

### BACKGROUND

Ambulatory medical devices (AMDs), including implantable, subcutaneous, wearable, or one or more other medical devices, etc., can monitor, detect, or treat, various conditions including, among other things, heart failure (HF), fibrillation, and myocardial infarction. Heart failure is a condition caused by impairment of the blood pumping action of the heart. Ambulatory medical devices can include sensors to sense physiological information from a patient and one or more circuits to detect one or more physiologic events using the sensed physiological information or transmit sensed physiologic information or detected physiologic events to one or more remote devices. Patient monitoring can provide early detection of worsening patient condition, including HF.

### SUMMARY

Systems and methods are disclosed for device-based detection of pulmonary hypertension of a patient. Pulmonary Hypertension (PH) is a condition of increased blood pressure in the arteries of the lungs. Detection of PH can be prognostic of heart failure.

Example 1 includes subject matter, such as an example ambulatory medical device (AMD), comprising an activity sensor configured to produce an activity signal representative of activity level of a patient, a heart sound sensor configured to produce a heart sound signal, and a control circuit operatively coupled to the activity sensor and the heart sound sensor. The control circuit is configured to detect a change in activity level of the patient using the activity signal, enable sensing of the heart sound signal in response to detecting the change in activity level, measure intensity of an S3 heart sound in the sensed heart sound signal, and produce an indication of PH when the measured intensity of the S3 heart sound exceeds a PH detection threshold intensity.

In Example 2, the subject matter of Example 1 optionally includes a control circuit configured to detect exercise of the patient using the activity signal, and enable the sensing of the heart sound signal in response to detecting the exercise.

In Example 3, the subject matter of one or both of Examples 1 and 2 optionally includes a control circuit configured to detect an increase in ambulatory activity of the patient using the activity signal, and enable the sensing of the heart sound signal in response to detecting the increase in ambulatory activity.

In Example 4, the subject matter of one or any combination of Examples 1-3 optionally includes a communication circuit configured to communicate information wirelessly with a second device and a control circuit configured to send a message to the second device indicative of PH in response to the measured intensity of the S3 heart sound exceeding the PH detection threshold intensity.

In Example 5, the subject matter of one or any combination of Examples 1-4 optionally includes at least one other physiologic sensor configured to produce at least one other sensed physiological signal that includes physiological information of the patient, and a control circuit configured to detect heart failure (HF) of the patient using the at least one other sensed physiological signal, and change at least one of sensitivity of the HF detection and specificity of the HF detection by the control circuit in response to the measured intensity of the S3 heart sound exceeding the PH detection threshold intensity.

In Example 6, the subject matter of Example 5 optionally includes a control circuit configured to activate the at least one other physiologic sensor in response to the measured intensity of the S3 heart sound exceeding the PH detection threshold intensity.

In Example 7, the subject matter of one or any combination of Examples 1-6 optionally includes at least one other physiologic sensor configured to produce at least one other sensed physiological signal that includes physiological information of the patient, and a control circuit configured to activate the at least one other physiologic sensor in response to the measured intensity of the S3 heart sound exceeding the PH detection threshold intensity, and generate the indication of PH using both the sensed heart sound signal and the at least one other sensed physiological signal.

In Example 8, the subject matter of Example 7 optionally includes at least one of a cardiac signal sensor configured to produce a sensed cardiac signal including heart rate information of the patient, a temperature sensor configured to produce temperature information of the patient, or a lung sound sensor configured to produce a sensed lung sound signal including lung sounds of the patient, and also optionally includes a control circuit configured to generate the indication of PH using both of the measured intensity of the S3 heart sound and the at least one of the heart rate information, the temperature information, or a lung sound of the patient.

Example 9 includes subject matter (such as a method of operating an AMD) or can be combined with one or any combination of Examples 1-8 to include such subject matter, comprising determining a perturbation in behavior of a patient using the AMD, activating sensing of a heart sound signal by the AMD in response to the detecting of the perturbation, measuring intensity of an S3 heart sound in the heart sound signal; and generating an indication of PH when the measured intensity of the S3 heart sound exceeds a PH detection threshold intensity.

In Example 10, the subject matter of Example 9 optionally includes detecting that the patient is exercising and activating the sensing of the heart sound signal in response to detecting the exercising.

In Example 11, the subject matter of one or both of Examples 9 and 10 optionally includes detecting a change in ambulatory daily activity of the patient, and activating the sensing of the heart sound signal in response to detecting the change in ambulatory daily activity of the patient.

In Example 12, the subject matter of one or any combination of Examples 9-11 optionally includes receiving information of medication dosing of the patient and monitoring the intensity of the S3 heart sound in association with the activity level of the patient is response to the receiving the medication dosing information.

In Example 13, the subject matter of one or any combination of Examples 9-12 optionally includes sending an alert of the PH to a second device.

In Example 14, the subject matter of one or any combination of Examples 9-13 optionally includes sensing at least one other sensed physiologic signal, wherein the least one sensed physiologic signal includes physiological information of the patient; monitoring, by the AMD, heart failure status of the patient using the at least one sensed physiological signal; and changing one or both of sensitivity and specificity of heart failure detection by the AMD in response to the generated indication of PH.

In Example 15, the subject matter of one or any combination of Examples 9-14 optionally includes activating at least one other physiologic sensor in response to the detecting the S3 heart sound to produce at least one sensed physiologic signal, wherein the least one sensed physiologic signal includes physiological information of the patient, and producing a composite health index using the heart sound signal and the at least one other sensed physiological signal, wherein the composite health index is indicative of heart failure status of the patient.

In Example 16, the subject matter of one or any combination of Examples 9-15 optionally includes activating at least one other physiologic sensor in response to detecting the S3 heart sound in the heart sound signal to produce at least one other sensed physiologic signal, wherein the least one other sensed physiologic signal includes physiological information of the patient; and generating the indication of PH using both the sensed heart sound signal and the at least one other sensed physiological signal.

In Example 17, the subject matter of Example 16 optionally includes the at least one sensed physiological signal includes physiological information of at least one of heart rate of the patient, temperature of the patient, or lung sound information of the patient; and generating the indication of PH using S3 heart sound information and the at least one of the heart rate of the patient, the temperature of the patient, or the lung sound information of the patient.

Example 18 includes subject matter (such as a medical device system) or can optionally be combined with one or any combination of Examples 1-27 to include such subject matter, comprising a signal receiver circuit configured to receive activity information of a patient and heart sound information of the patient, and a control circuit. The control circuit is configured to detect an increase in activity level of the patient using the activity information, measure intensity of an S3 heart sound in the heart sound information, and produce an alert of pulmonary hypertension (PH) as a function of the measured intensity of the S3 heart sound and the detected increase in activity level when the measured intensity of the S3 heart sound exceeds a PH detection threshold intensity.

In Example 19, the subject matter of Example 18 optionally includes an AMD including a heart sound sensor configured to produce the heart sound information, and a communication link to send the heart sound information to the control circuit, and the control circuit is configured to produce a composite health index using at least the activity information and the heart sound information, wherein the composite health index is indicative of heart failure status of the patient, detect worsening heart failure status using the composite index, and increase sensitivity to detecting the worsening heart failure status in response to the heart sound information received from the AMD indicating the measured intensity of the S3 heart sound exceeds the PH detection threshold intensity.

In Example 20, the subject matter of one or both of Examples 18 and 19 optionally includes a control circuit configured to receive medication dosing information, and monitoring the intensity of the S3 heart sound in association with the activity level of the patient is response to the receiving the medication dosing information.

These non-limiting examples can be included in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example patient management system.

FIG. 2 illustrates an example of an ambulatory medical device (AMD) that is implantable.

FIG. 3 illustrates an example of an AMD that is insertable.

FIG. 4 is a block diagram of an example of electronic circuits of an AMD.

FIG. 5 is a block diagram of an example of electronic circuits of an external device included in a patient management system.

FIG. 6 illustrates histograms of S3 heart sounds measured for heart failure patients.

FIG. 7 is a flow diagram of an example of a method of operating an AMD.

FIG. 8 is a block diagram of an example of a medical device system.

FIG. 9 is a flow diagram of an example of a method of operating a medical device system.

FIG. 10 is a block diagram of an example of a medical device system.

FIG. 11 illustrates a block diagram of an example machine upon which any one or more of the techniques discussed herein may perform.

### DETAILED DESCRIPTION

Ambulatory medical monitoring devices can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

The present inventors have recognized, among other things, systems, and methods to provide device-based detection of pulmonary hypertension of a patient. The device-based monitoring can improve the time to intervention to address patient heart failure.

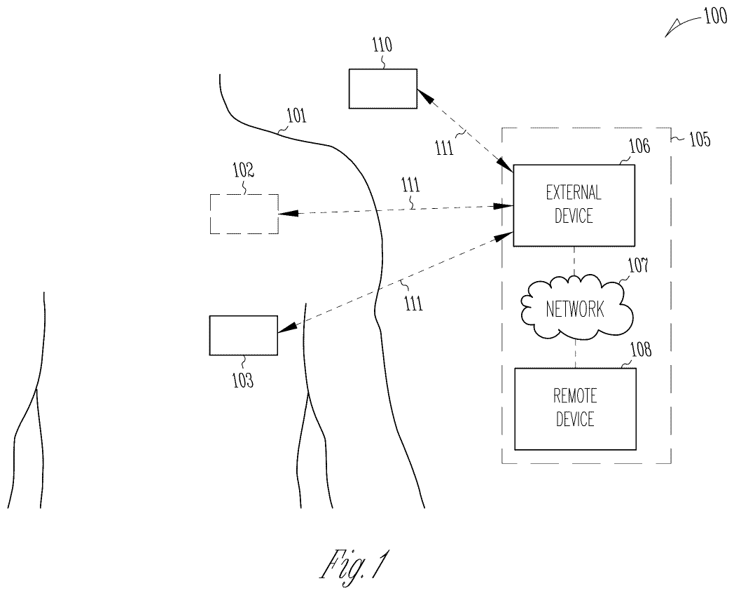

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more medical devices, an external system 105, and a communication link 111 providing for communication between the one or more ambulatory medical devices and the external system 105. The one or more medical devices can include an ambulatory medical device (AMD), such as an implantable medical device (IMD) 102, insertable cardiac monitor (ICM), a wearable medical device 103, or one or more other implantable, leadless, subcutaneous, external, wearable, or medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 of FIG. 1 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemakers can communicate between themselves, or one or more other implanted or external devices.

The IMD 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes, such as described herein. The implantable medical device 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the implantable medical device 102 or one or more of the other ambulatory medical devices, etc. In some examples, therapy can include CRT for rectifying dyssynchrony and improving cardiac function in heart failure patients. In other examples, the implantable medical device 102 can include a drug delivery system, such as a drug infusion pump to deliver drugs to the patient for managing arrhythmias or complications from arrhythmias, hypertension, hypotension, or one or more other physiologic conditions. In other examples, the implantable medical device 102 can include one or more electrodes configured to stimulate the nervous system of the patient or to provide stimulation to the muscles of the patient airway, etc.

The wearable medical device 103 can include one or more wearable or external medical sensors or devices (e.g., automatic external defibrillators (AEDs), Holter monitors, patch-based devices, smart watches, smart accessories, wrist-or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the implantable medical device 102 or one or more other ambulatory medical devices connected to the external system 105 via a communication link 111. In other examples, the IMD 102 can be connected to the wearable medical device 103, or the wearable medical device 103 can be connected to the external system 105, via the communication link 111. This can include, for example, programming the IMD 102 to perform one or more of acquiring physiologic data, performing at least one self-diagnostic test (such as for a device operational status), analyzing the physiologic data, or optionally delivering or adjusting a therapy for the patient 101. Additionally, the external system 105 can send information to, or receive information from, the IMD 102 or the wearable medical device 103 via the communication link 111. Examples of the information can include real-time or stored physiologic data from the patient 101, diagnostic data, such as detection of patient hydration status, hospitalizations, responses to therapies delivered to the patient 101, or device operational status of the implantable medical device 102 or the wearable medical device 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 602. 11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or both of the external device 106 and the remote device 108 can output the detected medical events to a system user, such as the patient or a clinician, or to a process including, for example, an instance of a computer program executable in a microprocessor or other processor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The system includes a therapy device 112 that can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 112. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

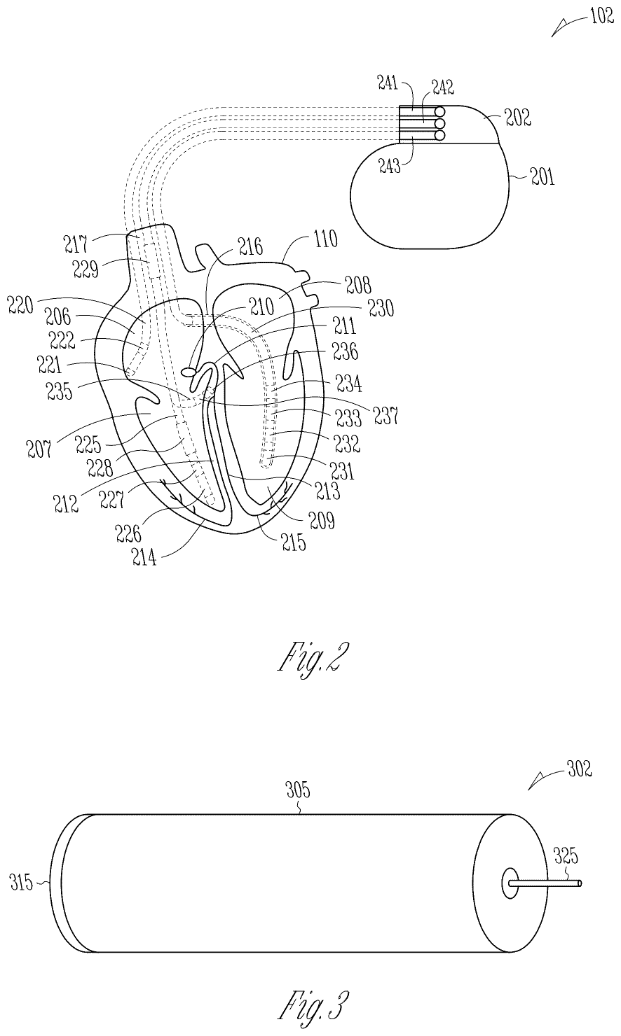

FIG. 2 illustrates an example of an ambulatory medical device that is an IMD 102. The IMD 102 is electrically coupled to a heart 110, such as through one or more leads coupled to the IMD 102 through one or more lead ports, such as first, second, or third lead ports 241, 242, 243 in a header 202 of the IMD 102. In an example, the IMD 102 can include an antenna, such as in the header 202, configured to enable communication with an external system and one or more electronic circuits in a hermetically sealed housing (CAN) 201. The IMD 102 illustrates an example medical device (or a medical device system) as described herein.

The IMD 102 may be an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the IMD 102 can include one or more electrodes or other sensors (e.g., a pressure sensor, an accelerometer, a gyroscope, a microphone, etc.) powered by a power source in the IMD 102. The one or more electrodes or other sensors of the leads, the IMD 102, or a combination thereof, can be configured detect physiologic information from, or provide one or more therapies or stimulation to, the patient.

The IMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN 201 such as for unipolar sensing of an electrogram or for delivering one or more pacing pulses. A defibrillation electrode (e.g., the first defibrillation coil electrode 228, the second defibrillation coil electrode 229, etc.) may be used together with the CAN 201 to deliver one or more cardioversion/defibrillation pulses.

In an example, the IMD 102 can sense impedance such as between electrodes located on one or more of the leads or the CAN 201. The IMD 102 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 102 can be configured to inject current between an electrode on one or more of the first, second, third, or fourth leads 220, 225, 230, 235 and the CAN 201, and to sense the resultant voltage between the same or different electrodes and the CAN 201.

The example lead configurations in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively, and a fourth lead 235 positioned in the RV 207 near the His bundle 211, between the AV node 210 and the right and left bundle branches 212, 213 and Purkinje fibers 214, 215. Each lead can be configured to position one or more electrodes or other sensors at various locations in or near the heart 110 to detect physiologic information or provide one or more therapies or stimulation.

The first lead 220, positioned in the RA 206, includes a first tip electrode 221 located at or near the distal end of the first lead 220 and a first ring electrode 222 located near the first tip electrode 221. The second lead 225 (dashed), positioned in the RV 207, includes a second tip electrode 226 located at or near the distal end of the second lead 225 and a second ring electrode 227 located near the second tip electrode 226. The third lead 230, positioned in the coronary vein 216 over the LV 209, includes a third tip electrode 231 located at or near the distal end of the third lead 230, a third ring electrode 232 located near the third tip electrode 231, and two additional electrodes 233, 234. The fourth lead 235, positioned in the RV 207 near the His bundle 211, includes a fourth tip electrode 236 located at or near the distal end of the fourth lead 235 and a fourth ring electrode 237 located near the fourth tip electrode 236. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 includes a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 211.

The IMD 102 can include a heart sound sensor to produce a heart sound signal. Heart sounds are recurring mechanical signals associated with cardiac vibrations or accelerations resulting from different valve closures over a cardiac cycle. Heart sounds can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4).

Valve closures and blood movement and pressure changes in the heart can cause accelerations, vibrations, or movement of the cardiac walls that can be detected using a heart sound sensor such as an accelerometer or a microphone, producing a heart sound signal. As opposed to detecting heart sounds using auscultation, device-based detection of heart sounds (e.g., with an accelerometer) can detect frequencies of heart sounds that wouldn't be detected by auscultation. In an example, heart sound signal portions, or values of respective heart sound signals for a cardiac interval, may be detected by comparison with a sensed cardiac signal. For instance, the value and timing of an S1 signal can be detected using an amplitude or energy of the heart sound signal occurring at or about the R wave of the cardiac interval. The S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles. For example, a heart sound parameter can include a composite S1 parameter representative of a plurality of S1 parameters, for example, over a certain time period (e.g., a number of cardiac cycles, a representative time period, etc.). In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform.

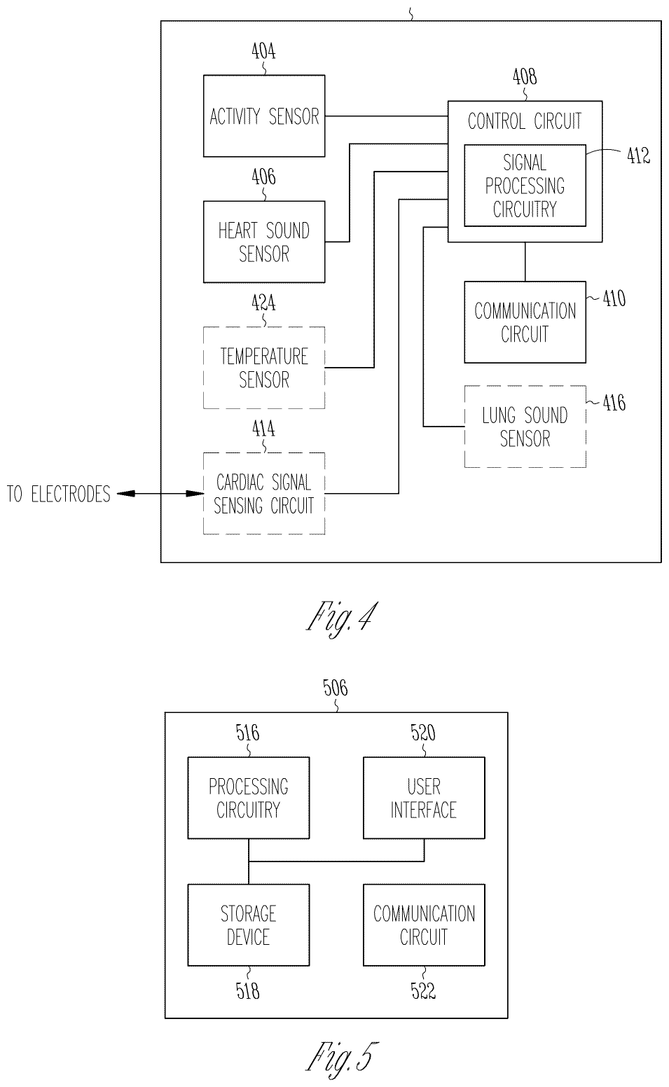

FIG. 3 illustrates portions of an example of a leadless ambulatory medical device (AMD) 302 that is insertable. In some examples, the AMD 302 is an insertable cardiac monitoring device (ICM). The AMD 302 includes a housing having an elongate cylindrical body 305. The AMD 302 can include one or more physiologic sensors to collect physiological information of the patient. In some examples, AMD includes a cardiac signal sensing circuit. The cardiac signal sensor produces sensed cardiac signals when connected to electrodes. The AMD 302 can include a first electrode incorporated into a pin 325 arranged at a first capped end of the housing, and a second electrode incorporated into a surface located at a second capped end 315. Portions of the pin 325 and the surface of the second capped end 315 are conductive and contact tissue and fluid. The cylindrical housing is electrically insulating. The cylindrical housing can include a ceramic or plastic. The electrodes can be used for both sensing and communication. In some examples, the electrodes located at the ends of AMD 302 are used for communication and a separate set of electrodes is included on the housing for cardiac signal sensing.

In some examples, the AMD 302 can include a heart sound sensor within the housing, such as an accelerometer or a microphone. In some examples, the AMD 302 can include an activity sensor such as an accelerometer or a tilt switch to detect activity such as movement of the patient. In some examples, the AMD 302 includes a temperature sensor that produces temperature information of the patient. In some examples, the AMD 302 includes a lung sound sensor such as a microphone. The lung sound sensor is configured to produce a sensed lung sound signal including lung sounds of the patient.

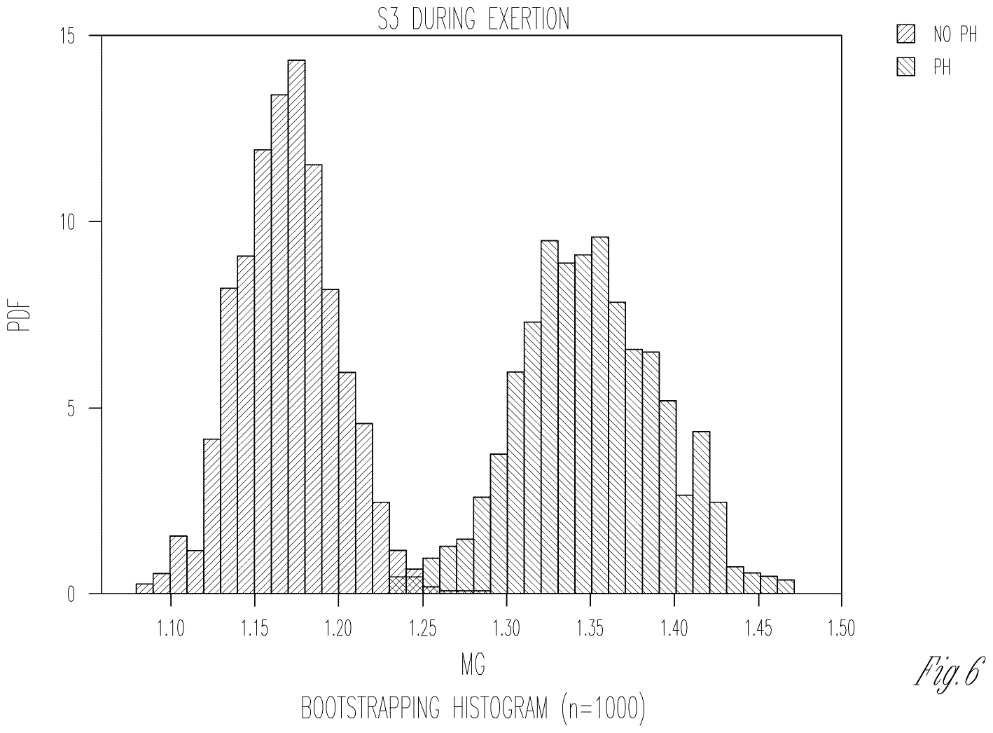

FIG. 4 is a block diagram of an example of portions of electronic circuits of an AMD 402. The AMD 402 may be implantable or insertable. The AMD 402 includes an activity sensor 404, a heart sound sensor 406, a communication circuit 410, and a control circuit 408. The activity sensor 404 produces an activity signal representative of activity level of a patient, and the heart sound sensor 406 produces a heart sound signal that include electrical representations of heart sounds of the patient. The communication circuit 410 can be used to communicate information wirelessly with a separate device, such as by using near-field inductive wireless signals or far-field radio-frequency signals.

The control circuit 408 may include a digital signal processor, application specific integrated circuit (ASIC), field programmable gate array (FPGA), microprocessor, or other type of processor, interpreting or executing instructions in software or firmware. In some examples, the control circuit 408 may include a state machine or sequencer that is implemented in hardware circuits. The control circuit 408 may include any combination of hardware, firmware, or software. The control circuit 408 includes electronic circuitry (e.g., signal processing circuitry 412) to perform the functions described herein. A circuit may include software, hardware, firmware, or any combination thereof. For example, the circuit may include instructions in software executing on the control circuit 408. Multiple functions may be performed by one or more circuits of the control circuit 308. The AMD 402 may include other physiologic sensors such as a temperature sensor 424, a cardiac signal sensing circuit 414, and a lung sound sensor 416 for example.

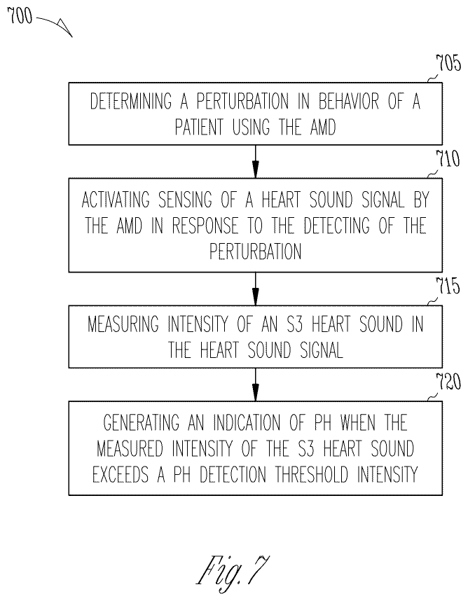

FIG. 5 is a block diagram of an example of portions of a device 506 included in an external system (e.g., external system 105 in FIG. 1). The device 506 may be either an external device of the external system (e.g., external device 106 in FIG. 1) or a remote device of the external system (e.g., remote device 108 in FIG. 1). The device 506 includes a storage device 518 and processing circuitry 516. In some examples, the device 506 includes a user interface 520. Processing circuitry 516 may be implemented using an application-specific integrated circuit (ASIC) constructed to perform one or more functions or a general-purpose circuit programmed to perform the functions. A general-purpose circuit can include, among other things, a microprocessor or a portion thereof, a microcontroller or portions thereof, and a programmable logic circuit or a portion thereof. The storage device 518 may be a memory integral to the processing circuitry 516, or a separate memory device.

The device 506 includes a communication circuit 522 to communicate information with another device. Communication circuit 522 may communicate information wirelessly with the AMD 402 of FIG. 4 using near-field inductive wireless signals or far-field radio-frequency signals. The device 506 can be used to program pacing therapy parameters and other information in the AMD 402. In some examples, the device 506 is remote from the AMD 402 and the communication circuit 522 communicates information over a network (e.g., a cellular phone network or the Internet) to another device that communicates information wirelessly with the AMD 402.

Increased left ventricular (LV) filling pressure is a distinctive characteristic of heart failure (HF). Within the closed hemodynamic system of a patient, increased LV filling pressure results in elevated pressures in the left atrium (LA) and in the pulmonary venous vasculature. Patients with HF frequently develop pulmonary hypertension (PH). HF patients account for a majority of patients with PH, and HF is the leading cause of PH. Thus, detection of PH in patients is highly prognostic of HF. Current PH detection techniques involve right heart catheterization (RHC) to test pulmonary artery pressure (PAP) and function of the right ventricle (RV). Doppler Echo can be used to estimate pulmonary artery systolic pressure (PASP) using tricuspid valve velocity. However, RHC and Doppler Echo are costly procedures done in a clinical setting and are done infrequently. The burden of RHC or Doppler Echo precludes frequent or continuous monitoring of the patient to detect PH. Thus, improvements in monitoring of PH are desirable.

FIG. 6 illustrates histograms of S3 heart sounds measured for HF patients with PH and HF patients without PH. The S3 heart sounds measured during exertion of the patient. The histograms are bootstrapping histograms with the horizontal axis being milli-G's (mG) and the vertical axis being a probability distribution function (PDF). The histograms show that the S3 heart sounds during exertion for patients with PH have a higher intensity than for patients without PH. FIG. 6 shows that the S3 heart sound can be used as a biomarker for PH detection in patients with HF.

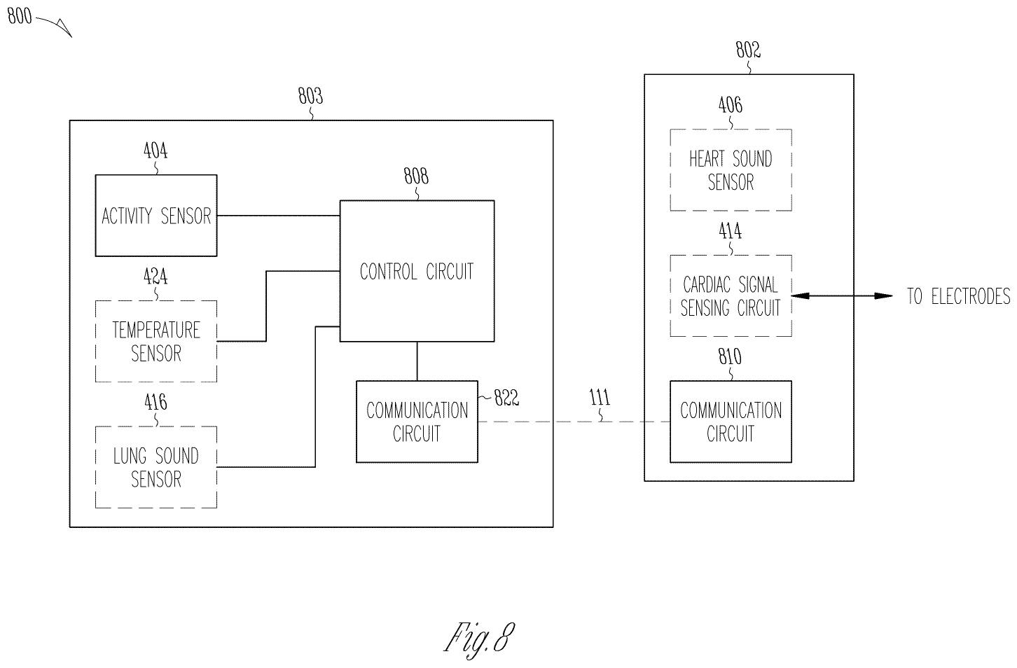

FIG. 7 is a flow diagram of an example of a method 700 of operating an AMD (e.g., any of the AMDs described herein regarding FIGS. 1-4). At block 705, the AMD determines a perturbation in the behavior of the patient using the AMD, and at block 710, the AMD activates sensing of a heart sound signal by the AMD in response to the perturbation in behavior of the patient.

For instance, the perturbation may be an increase in activity of the patient. The AMD may include an activity sensor, a heart sound sensor, and a control circuit (e.g., activity sensor 404, heart sound sensor 406, and control circuit 408 in FIG. 4). The activity sensor produces an activity signal representative of activity level of the patient. The control circuit 408 detects an increase in activity of the patient using the activity signal and activates the heart sound sensor 406 (e.g., by allowing power to be supplied to the sensor or logically enabling use of the output of the sensor) to produce a sensed heart sound signal during the detected increased activity. In some examples, the control circuit activates the heart sound sensor 406 when detecting that the patient is exercising based on the activity signal, but the control circuit may activate the heart sound sensing in response to any detected increase in ambulatory daily activity of the patient.

When the heart sound sensor is activated, the control circuit 408 detects an S3 heart sound in the sensed heart sound signal. The AMD 402 may include signal processing circuitry 412 to detect the S3 heart sound. In certain examples, the AMD 402 includes the cardiac signal sensing circuit 414. The cardiac signal sensing circuit 414 senses cardiac signals when connected to sensing electrodes. The signal processing circuitry 412 may identify an R-wave in a sensed cardiac signal associated with the sensed heart sound signal. The signal processing circuitry may identify an S1 or S2 heart sound from a time relationship to the R-wave and identify the S3 heart sound from its time relationship to the S1 or S2 heart sound.

At block 715, the control circuit measures the intensity of the S3 heart sound detected in the heart sound signal. The measure of intensity may be the magnitude of the S3 heart sound. At block 720, the control circuit generates an indication of PH when the measured intensity of the S3 heart sound exceeds a PH detection threshold intensity.

Returning to FIG. 6, it can be seen that the intensity of the S3 heart sounds during exertion is greater for patients having PH than for patients not having PH. For the example data in FIG. 6, the control circuit may generate the indication of PH when the magnitude of the S3 heart sound is greater than or equal to 1.3 mGs. The magnitude value of 1.3 mGs is only an example, and the magnitude value may be different for a different patient population. In some examples, the control circuit may generate the indication of PH according to a change in intensity of the S3 heart sound. For instance, the AMD may store a baseline value of intensity of the S3 heart sound, and the control circuit may generate the indication of PH when the measured intensity of the S3 heart sound changes from the baseline intensity by the PH detection threshold intensity.

In some examples, the indication of PH may be an alert sent to a second device. For instance, the AMD 402 may store the indication of PH in memory. The second device may be an external device (e.g., external device 506) that communicates with the AMD 402. When PH of the patient is detected by the AMD 402, this information can be sent to the external device 506 the next time the external interrogates the AMD for information. The external device 506 may display the alert of detection of PH of the patient to a user. An RHC or Doppler Echo procedure may then be scheduled and performed to confirm the PH diagnosis by the AMD 402, thereby reducing the time to diagnosis of PH over the approach of waiting for condition of the patient to worsen before performing an RHC or Doppler Echo procedure. Reducing the time to diagnosis of PH may reduce emotional uncertainty in the patient due to changing symptoms, reduce the use of health care resources, and enable treatment at an earlier stage of PH when therapies to address PH may be more effective.

The AMD 402 may transition from a lower power mode to a higher power mode when collecting the heart sound information. The higher power mode can include one or more of: enabling one or both of the cardiac signal sensing circuit 414 and the heart sound sensor 406, increasing a sensing frequency or a sensing or storage resolution, increasing an amount of data to be collected, communicated (e.g., to a second medical device, etc.), or stored, triggering storage of currently available information or increasing the storage capacity or time period of a loop recorder, or otherwise altering device behavior to capture additional or higher-resolution physiologic information or perform more processing, etc. After the sensing, recording, and transmitting of information, the AMD 402 returns to the lower power mode.

In some examples, the control circuit 408 may activate one or more other physiologic sensors of the AMD in response to the measured intensity of the S3 heart sound exceeding the PH detection threshold intensity. The other physiologic sensor or sensors may each produce a physiological signal that includes physiological information of the patient. The control circuit 408 may use the sensed heart sound signal to detect the PH, use one or more other physiological signals to confirm PH, and generate the indication of PH when the detection is confirmed. The additional physiological information can also be used to further diagnose the condition of the patient.

In some examples, the AMD 402 includes the cardiac signal sensing circuit 414 and the control circuit 408 can determine heart rate of the patient using a cardiac signal sensed by the cardiac signal sensing circuit 414. The AMD 402 may also include temperature sensor 424 that produces temperature information of the patient. The control circuit 408 may monitor nighttime heart rate (nHR) and temperature of the subject for a higher NHR and temperature. The additional information of the nHR and temperature may lead to a diagnosis of the patient potentially having an infection that causes the increased intensity of the S3 heart sound. The additional information may be sent to the second device (e.g., external device 506 in FIG. 5) and presented to the user using the display.

In some examples, the AMD 402 includes a lung sound sensor 416 that produces a lung sound signal biomarker representative of lung sounds of the patient. The additional lung sound information may confirm the PH detection, or lead to a diagnosis of worsening chronic obstructive pulmonary disease (COPD), or other condition.

The biomarker of PH can be used in managing patients with HF. The biomarker of PH can be monitored with changes in patient treatment to see if the treatment is working or not. For instance, the treatment may involve a medication change (e.g., the patient may be prescribed a vasodilator or a change in dosage of a vasodilator). The biomarker of PH can used to determine if the medication change is working. The control circuit may initiate the monitoring of the S3 heart sound intensity changes in response to the medication change to see if the S3 heart sound intensity changes differently with increase in activity.

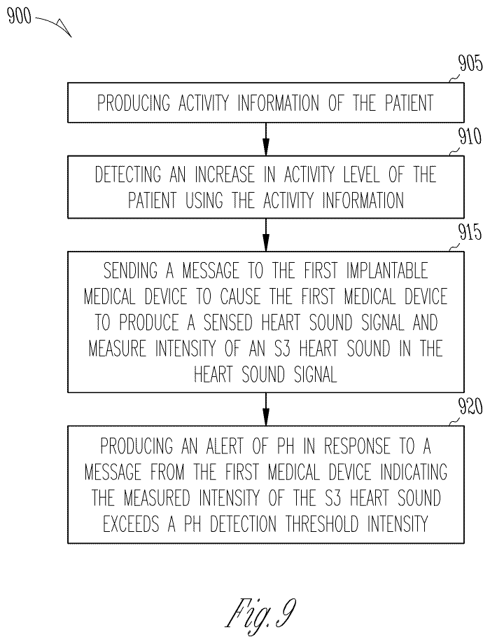

FIG. 8 is a block diagram of an example of a medical device system 800. The system 800 includes an AMD 802 that may be implantable or insertable. The AMD 802 includes the heart sound sensor 406 and a communication circuit 410. The AMD 802 may also include a cardiac signal sensing circuit 414. The system 800 includes a second device 803 that may be a wearable medical device. In certain examples, the second device 803 is a personal device of the patient's such as a smartwatch or smartphone. The second device 803 includes the activity sensor 404, a control circuit 808, and a second communication circuit 822 to communicate with the AMD 802 via a communication link 111. The second device 803 may include additional physiologic sensors such as, among other things, a temperature sensor 424 and a lung sound sensor 416.

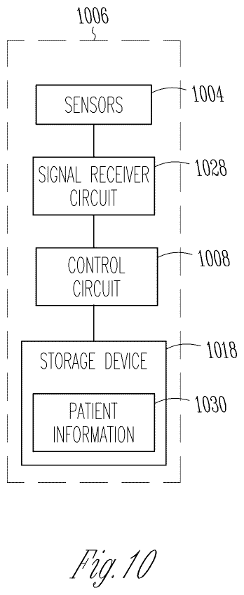

FIG. 9 is a flow diagram of an example of a method 900 of operating a medical device system such as the medical device system 800 of FIG. 8. At block 905, activity information of a patient is produced using the activity sensor 404. At block 910, the control circuit 808 detects an increase in activity level of the patient using the activity information. At block 915, the control circuit 808 sends a message (e.g., a command message or other prompt) to the AMD 802 to cause the AMD 802 to produce the heart sound signal and measure the intensity of an S3 heart sound in the sensed heart sound signal.

At block 920, the control circuit 808 produces an alert of PH in response to a message from the AMD 802 indicating when the measured intensity of the S3 heart sound exceeds a PH detection threshold intensity. In certain examples, the message from the AMD 802 includes a measure of S3 heart sound intensity of the S3 heart sound and the control circuit 808 compares the measure of S3 heart sound intensity to the PH detection threshold intensity to detect PH of the patient. In certain examples, the AMD 802 compares the measure of S3 heart sound intensity to the PH detection threshold intensity to detect PH of the patient, and the message from the AMD 802 indicates PH when the measured intensity exceeds the PH detection threshold intensity.

In some examples, the second device 803 produces a composite health index using at least the activity information and heart sound information. The composite health index is indicative of heart failure status of the patient. The composite health index can be determined using physiologic information of the patient obtained from multiple physiologic sensors and from other information of the patient's condition.

One or more composite health indexes can be determined, in certain examples, as a function of different physiologic information of the patient or various combinations thereof. In certain examples, a composite health index can be a device-based index, without input of clinical information about the patient, such as clinician diagnosis or determination of risk, patient history, patient age, comorbidities, prior hospitalization, type of implanted device, etc. In other examples, the composite health index can be a combination of a device-based and clinical-based mortality risk index, including or taking into account clinical information about the patient, such as clinician diagnosis or determination of risk, patient history, patient age, comorbidities, prior hospitalization, type of implanted device, etc. In an example, separate determinations can be made for different combinations of clinical information.

One example of a composite health index is a HeartLogic™ index, a HeartLogic™ in-alert time, or one or more other composite measurements or measures thereof. The HeartLogic™ index is a composite measurement from multiple ambulatory sensors, including S1 and S3 heart sounds, thoracic impedance, activity information, respiration information, and nHR, indicative of a heart failure status, a risk of heart failure event, or a worsening of the heart failure status or risk of heart failure event in the patient over time. The HeartLogic™ in-alert time is a measure of time that the HeartLogic™ index is above an alert threshold.

For example, if the risk stratifier is low, or below a first threshold, the HeartLogic™ index can be determined using a first combination of physiologic information. If the risk stratifier is high, or above a second threshold, the HeartLogic™ index can be determined using the first combination of physiologic information and a second combination of physiologic information, including additional information than included in the first combination. If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination).

In an example, the HeartLogic™ index and in-alert time can include worsening heart failure or physiologic event detection, including risk indication or stratification, such as that disclosed in the commonly assigned An et al. U.S. Pat. No. 9,568,266 entitled “RISK STRATIFICATION BASED HEART FAILURE DETECTION ALGORITHM,” or in the commonly assigned An et al. U.S. Pat. No. 9,422,464 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,460,377 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,496 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

The second device 803 in FIG. 8 may determine the composite health index, or the composite health index may be determined by an external device (e.g., external device 106 in FIG. 1) or a remote device (e.g., remote device 108 in FIG. 1). A worsening heart failure status of the patient may be detected using the composite index. One or both of sensitivity and specificity of HF detection using the composite health index may be changed in response to the generated indication of PH. For instance, the second device 803 may increase sensitivity of the composite health index to detecting the worsening heart failure status in response to the message from the AMD 802 indicating the measured intensity of the S3 heart sound exceeds the PH detection threshold intensity.

The changing of the sensitivity of the composite health index may include changing one or more sensor index time constants, or increasing or decreasing a threshold for generating an alert of worsening HF. In certain examples, some physiologic sensors are given a primary status for contributing to the HF detection, and some are given a secondary status. In response to the indication of PH based on the S3 heart sound during exertion of the patient, the indication from some secondary sensors may be reassigned a primary status. In some examples, sensors that were given a primary status may be reassigned to a secondary status. For instance, activity contribution to the composite health index may be reassigned to a secondary status because patients with PH often become less active. The biomarker of PH can be included in the composite health index to improve device-based monitoring of HF status of the patient.

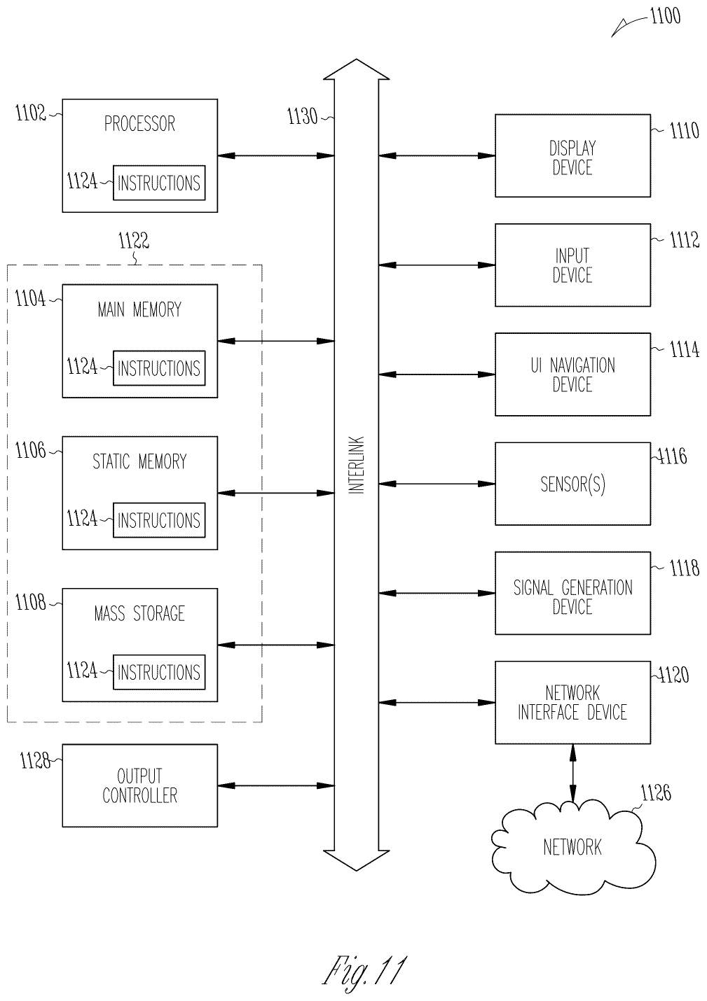

FIG. 10 is a block diagram of portions of another example of a medical device system. The system 1006 includes a signal receiver circuit 1028, a control circuit 1008, and a storage device 1018. The signal receiver circuit 1028 receives activity information of a patient. In some examples, the system 1006 is an external system and the signal receiver circuit 1028 receives activity information from a separate device (e.g., an AMD). In some examples, the system 1006 includes sensors 1004 such as an activity sensor 404.

The signal receiver circuit 1028 also receives heart sound information of the patient. In some examples, the signal receiver circuit 1028 receives a heart sound signal from a separated device (e.g., an AMD). In some examples, the signal receiver circuit 1028 receives heart sound information processed by the separate device (e.g., via a communication link with the separate device). In some examples, the system 1006 includes a heart sound sensor 406. In some examples, the sensors 1004 include an accelerometer and the signal receiver circuit 1028 is configured to process accelerometer signals to determine the heart sound information.

The storage device 1018 stores patient information 1030. The patient information 1030 may include medication dosing information and clinical information for the patient. In some examples, the patient information is stored in a cloud-based server, and the system 1006 retrieves the patient information from the server. The control circuit 1016 may be implemented using an ASIC constructed to perform one or more functions or a general-purpose circuit programmed to perform the functions. The control circuit 1008 is configured to detect an increase in activity level of the patient using the activity information and measure intensity of an S3 heart sound in the heart sound information. The control circuit 1008 produces an alert of PH as a function of the measured intensity of the S3 heart sound and the detected change in activity level when the measured intensity of the S3 heart sound exceeds a PH detection threshold intensity. The system 1006 may present the alert to the user or send the alert to the cloud-based server.

In some examples, the control circuit 1008 produces a composite health index using at least the activity information and the heart sound information. In some examples, the control circuit 1008 produces the composite health index using the patient information 1030, the activity information, and the heart sound information. The system 1006 may use the composite index to detect one or more of worsening heart failure status or worsening heart failure risk status. In response to worsening status of the patient, the system 1006 may increase sensitivity to detecting the worsening status of the patient in response to the heart sound information received from the AMD indicating the measured intensity of the S3 heart sound exceeds the PH detection threshold intensity.

FIG. 11 is a block diagram of an example machine 1100 upon which any one or more of the techniques (e.g., methodologies) discussed herein may perform. Portions of this description may apply to the computing framework of one or more of the medical devices described herein, such as the ambulatory medical device, the wearable medical device, the external programmer, the remoter server, etc., in computing a composite health index. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1100. Circuitry (e.g., signal processing circuitry, etc.) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1100 (e.g., computer system) may include a hardware processor 1102 (e.g., a central processing unit (CPU), a graphics processing unit (GPU), a hardware processor core, or any combination thereof), a main memory 1104, a static memory 1106 (e.g., memory or storage for firmware, microcode, a basic-input-output (BIOS), unified extensible firmware interface (UEFI), etc.), and mass storage 1108 (e.g., hard drive, tape drive, flash storage, or other block devices) some or all of which may communicate with each other via an interlink 1130 (e.g., bus). The machine 1100 may further include a display unit 1110, an input device 1112 (e.g., a keyboard), and a user interface (UI) navigation device 1114 (e.g., a mouse). In an example, the display unit 1110, input device 1112, and UI navigation device 1114 may be a touch screen display. The machine 1100 may additionally include a signal generation device 1118 (e.g., a speaker), a network interface device 1120, and one or more sensors 1116, such as a global positioning system (GPS) sensor, compass, accelerometer, or one or more other sensors. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 1102, the main memory 1104, the static memory 1106, or the mass storage 1108 may be, or include, a machine-readable medium 1122 on which is stored one or more sets of data structures or instructions 1124 (e.g., software) embodying or utilized by any one or more of the techniques or functions described herein. The instructions 1124 may also reside, completely or at least partially, within any of registers of the hardware processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine-readable medium 1122. While the machine-readable medium 1122 is illustrated as a single medium, the term “machine-readable medium” may include a single medium or multiple media (e.g., a centralized or distributed database, and/or associated caches and servers) configured to store the one or more instructions 1124.

The term “machine-readable medium” may include any medium that is capable of storing, encoding, or carrying instructions for execution by the machine 1100 and that cause the machine 1100 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1100, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. An ambulatory medical device, the device comprising:
an activity sensor configured to produce an activity signal representative of activity level of a patient;
a heart sound sensor configured to produce a heart sound signal; and
a control circuit operatively coupled to the activity sensor and the heart sound sensor, wherein the control circuit is configured to:
detect a change in activity level of the patient using the activity signal;
enable sensing of the heart sound signal in response to detecting the change in activity level;
measure intensity of an S3 heart sound in the sensed heart sound signal; and
produce an indication of pulmonary hypertension (PH) when the measured intensity of the S3 heart sound exceeds a PH detection threshold intensity.

2. The device of claim 1, wherein the control circuit is configured to:
detect exercise of the patient using the activity signal; and
enable the sensing of the heart sound signal in response to detecting the exercise.

3. The device of claim 1, wherein the control circuit is configured to:
detect an increase in ambulatory activity of the patient using the activity signal; and
enable the sensing of the heart sound signal in response to detecting the increase in ambulatory activity.

4. The device of claim 1, including:
a communication circuit configured to communicate information wirelessly with a second device; and
wherein the control circuit is configured to send a message to the second device indicative of PH in response to the measured intensity of the S3 heart sound exceeding the PH detection threshold intensity.

5. The device of claim 1, including:
at least one other physiologic sensor configured to produce at least one other sensed physiological signal that includes physiological information of the patient; and
wherein the control circuit is configured to:
detect heart failure (HF) of the patient using the at least one other sensed physiological signal; and
change at least one of sensitivity of the HF detection and specificity of the HF detection by the control circuit in response to the measured intensity of the S3 heart sound exceeding the PH detection threshold intensity.

6. The device of claim 5, wherein the control circuit is configured to activate the at least one other physiologic sensor in response to the measured intensity of the S3 heart sound exceeding the PH detection threshold intensity.

7. The device of claim 1, including:
at least one other physiologic sensor configured to produce at least one other sensed physiological signal that includes physiological information of the patient; and
wherein the control circuit is configured to:
activate the at least one other physiologic sensor in response to the measured intensity of the S3 heart sound exceeding the PH detection threshold intensity; and
generate the indication of PH using both the sensed heart sound signal and the at least one other sensed physiological signal.

8. The device of claim 7, wherein the at least one other physiologic sensor includes at least one of:
a cardiac signal sensor configured to produce a sensed cardiac signal including heart rate information of the patient, a temperature sensor configured to produce temperature information of the patient, or a lung sound sensor configured to produce a sensed lung sound signal including lung sounds of the patient; and
wherein the control circuit is configured to generate the indication of PH using both of the measured intensity of the S3 heart sound and the at least one of the heart rate information, the temperature information, or a lung sound of the patient.

9. A method of operating an ambulatory medical device (AMD), the method comprising:
determining a perturbation in behavior of a patient using the AMD;
activating sensing of a heart sound signal by the AMD in response to the determining the perturbation;
measuring intensity of an S3 heart sound in the heart sound signal; and
generating an indication of pulmonary hypertension (PH) when the measured intensity of the S3 heart sound exceeds a PH detection threshold intensity.

10. The method of claim 9,
wherein the determining the perturbation in behavior of the patient includes detecting that the patient is exercising; and
wherein the activating the sensing of the heart sound signal includes activating the sensing of the heart sound signal in response to detecting the exercising.

11. The method of claim 9,
wherein the detecting the perturbation in behavior of the patient includes detecting a change in ambulatory daily activity of the patient; and
wherein the activating the sensing of the heart sound signal includes activating the sensing of the heart sound signal in response to detecting the change in ambulatory daily activity of the patient.

12. The method of claim 9, including:
receiving information of medication dosing of the patient; and
monitoring the intensity of the S3 heart sound in association with the activity level of the patient is response to the receiving the medication dosing information.

13. The method of claim 9, wherein the generating the indication of PH includes sending an alert of the PH to a second device.

14. The method of claim 9, including:
sensing at least one other sensed physiologic signal, wherein the least one sensed physiologic signal includes physiological information of the patient;
monitoring, by the AMD, heart failure status of the patient using the at least one sensed physiological signal; and
changing one or both of sensitivity and specificity of heart failure detection by the AMD in response to the generated indication of PH.

15. The method of claim 9, including:
activating at least one other physiologic sensor in response to the detecting the S3 heart sound to produce at least one sensed physiologic signal, wherein the least one sensed physiologic signal includes physiological information of the patient; and
producing a composite health index using the heart sound signal and the at least one other sensed physiological signal, wherein the composite health index is indicative of heart failure status of the patient.

16. The method of claim 9, including:
activating at least one other physiologic sensor in response to detecting the S3 heart sound in the heart sound signal to produce at least one other sensed physiologic signal, wherein the least one other sensed physiologic signal includes physiological information of the patient; and
wherein the generating the indication of PH includes generating the indication of PH using both the sensed heart sound signal and the at least one other sensed physiological signal.

17. The method of claim 16,
wherein the at least one sensed physiological signal includes physiological information of at least one of heart rate of the patient, temperature of the patient, or lung sound information of the patient; and
wherein the generating the indication of PH includes generating the indication of PH using S3 heart sound information and the at least one of the heart rate of the patient, the temperature of the patient, or the lung sound information of the patient.

18. A medical device system, the system comprising:
a signal receiver circuit configured to receive activity information of a patient and heart sound information of the patient; and
a control circuit configured to:
detect an increase in activity level of the patient using the activity information;
measure intensity of an S3 heart sound in the heart sound information; and
produce an alert of pulmonary hypertension (PH) as a function of the measured intensity of the S3 heart sound and the detected increase in activity level when the measured intensity of the S3 heart sound exceeds a PH detection threshold intensity.

19. The medical device system of claim 18, including:
an ambulatory medical device (AMD) including:
a heart sound sensor configured to produce the heart sound information; and
a communication link to send the heart sound information to the control circuit; and
wherein the control circuit is configured to:
produce a composite health index using at least the activity information and the heart sound information, wherein the composite health index is indicative of heart failure status of the patient;
detect worsening heart failure status using the composite index; and
increase sensitivity to detecting the worsening heart failure status in response to the heart sound information received from the AMD indicating the measured intensity of the S3 heart sound exceeds the PH detection threshold intensity.

20. The medical device system of claim 18, wherein the control circuit is configured to:
receive medication dosing information; and
initiate monitoring the intensity of the S3 heart sound in association with the activity level of the patient is response to the receiving the medication dosing information.
